(19) United States
(12) Patent Application Publication
(10) Pub. No.: US 2022/0202728 A1
(43) Pub. Date: Jun. 30, 2022

(54) METHODS FOR PRODUCING A PHARMACEUTICAL CARRIER

(71) Applicant: NOVARTIS AG, Basel (CH)

(72) Inventors: Florian BECK, Hallau (CH); Hans de Waard, Basel (CH); Sarah Gold, Frick (CH); Stefan Hirsch, Lörrach (DE); David Hook, Rheinfelden (CH); Nikhil Kavimandan, Plainfield, NJ (US); Markus Krumme, Allschwil (CH); Steffen Lang, Reinach (CH); Detlef MOLL, Hallau (CH); Siddharthya Mujumdar, Roschenz (CH); Anh-Thu Nguyen-Trung, Kembs (FR); Joerg Ogorka, Reinach (CH); Norbert Rasenack, Weil am Rhein (DE); Maxime Thomas-Schrapp, Basel (CH); Raphael TOBLER, Hallau (CH); Patrick Tritschler, Freiburg (DE)

(21) Appl. No.: 17/600,318

(22) PCT Filed: Apr. 1, 2020
(86) PCT No.: PCT/IB2020/053084

§ 371 Date: Sep. 30, 2021

Related U.S. Application Data
(60) Provisional application No. 62/828,697, filed on Apr. 3, 2019.

(51) Int. Cl. A61K 9/48; A61K 47/32; A61K 47/36; A61K 47/12; A61K 47/10

(52) U.S. Cl. CPC A61K 9/4816; A61K 47/32; A61K 47/10; A61K 47/12; A61K 47/36

(57) ABSTRACT

A formulation for injection moulding of a pharmaceutical carrier comprising 27-85% (w/w) of polyvinyl alcohol, and 10-60% (w/w) of a disintegration aid selected from maize starch, wheat starch, and combinations thereof; and optionally one or more excipients.

The present invention relates to a formulation for injection moulding of a pharmaceutical carrier used to enclose a pharmaceutical composition.

### BACKGROUND OF THE INVENTION

Two common dosage forms used to administer orally solid pharmaceutical compositions are filled hard capsules and compressed tablets. Hard capsules are typically made using gelatin. A common production method is to form the two parts by dipping stainless steel pins into a gelatin solution. The capsule halves are then stripped from the pins and trimmed before being joined to make each capsule. An alternative method of manufacture which can allow for more complex geometries is to use injection moulding.

The composition of traditional capsules is limited to polymers which have suitable rheological and film forming properties when dispersed in water. Injection moulding however, is a hot melt process, which necessitates very different material properties. This presents both an opportunity to move away from traditional capsule materials such as gelatin (animal derived, mechanical properties dependent on environmental conditions) and HPMC (dissolution lag time) and a challenge as the injection moulding process is very demanding with respect to required material properties. The materials must be thermally stable during the process, have good melt flow properties—particularly under high shear conditions, be flexible enough when cooled to be ejected from the machine and for this application be mechanically strong to enable pharmaceutical processing and dissolve quickly in water. In addition the material must be suitable for human consumption and be approved for pharmaceutical use.

GB2501607B describes a melt-processable, water soluble polymer composition comprising PVOH and 15-75% by weight of a hygroscopic salt. The composition is suggested for moulding thin-walled articles having a thickness of less than 200 microns. However, the process parameters required for injection moulding described in GB2501607B are expected to cause thermal degradation of the polymer and the addition of a hygroscopic salt will lead to high moisture sensitivity and softening resulting in risk of carrier opening under the stress of bulk handling and storage.

### SUMMARY OF THE INVENTION

We have developed a new formulation for injection moulding of pharmaceutical carriers. In particular, the high performance of the formulation in the injection moulding process enables flexibility in design of the carriers allowing for robust manufacture of design features with very small dimensions—traditionally a challenge in injection moulding. The formulation allows preparing pharmaceutical carriers comprising very fine and thin design details by injection moulding, which pharmaceutical carriers maintain stability of the pharmaceutically active agent during storage. At the same time, the formulation provides the pharmaceutical carrier with good dissolution rates.

In addition, we have developed a novel pharmaceutical dosage form, (also referred to herein as Prescido™) which is designed to have the functionality of a standard pharmaceutical capsule while maintaining the patient appeal of a tablet. The carriers described herein are manufactured via a precision injection moulding process, using a formulation designed to perform well in thermal processes, such as the formulation of the invention. Design & manufacturing features together with their benefits include, inter alia, thin wall sections (fast carrier disintegration times in aqueous media), small snap close features (tight closure prevents opening of carrier during transport and limits tampering of carrier contents), numbering of cavities (traceability and sorting of parts before use) and high weight & dimension precision (robust handling processes).

Accordingly, the present invention is directed to a formulation for injection moulding of a pharmaceutical carrier, wherein the formulation comprises 27-85% (w/w) of polyvinyl alcohol (in particular polyvinyl alcohol (4-88)), and 10-60% (w/w) of a disintegration aid selected from maize starch and wheat starch).

In embodiments, the formulation further comprises 0.3-3.0% (w/w) of a lubricant (in particular stearic acid), and/or 5-14% (w/w) of a process aid (in particular propan-2-glycol). The process aid allows processing the formulation at lower temperatures, thereby reducing the risk of thermal degradation. At the same time, increasing amounts yield a higher flexibility of the pharmaceutical carrier prepared from the formulation, such that the carriers open with only small amounts of applied force. We found that 5-14% of the processing aid is optimal for a reasonable process. The formulation may also comprise one or more excipients, as further defined below.

Hence, the formulation can be advantageously applied in a method for producing a pharmaceutical carrier comprising the steps of melting a formulation of the present disclosure and injecting the melt into a mould, and optionally cooling the injected melt and optionally ejecting the moulded material. The method may comprise a further step of sorting the carrier parts by mould cavity. In preferred embodiments, the lid part and the bottom part are connected to each other by a complementary closing mechanism, in particular wherein the closing mechanism comprises a first snap part which projects from the bottom part so as to face and to interact with a second snap part which projects from the lid part.

In addition to having excellent thermal processing properties, the formulation developed imparts a number of benefits to the carriers compared to traditional capsules, such as, for example, and very fast dissolution (rapid carrier rupture in aqueous media).

Accordingly, in still another aspect, the present invention relates to a pharmaceutical carrier, produced by the method of the present disclosure using the formulation of the present disclosure, comprising a lid part and a bottom part wherein at least one of the lid part and the bottom part has a first wall section (26, 30) with a thickness of 180-220 μm, preferably 185-215 μm, even more preferably 190-210 μm, even more preferably 195-205 μm, and most preferably about 200 μm, and a second wall section with a thickness of 350-450 μm, preferably 375-425 μm, more preferably 390-410 μm, and most preferably about 400 μm.

The first wall section of the lid part may define an entire top portion of the lid part. Alternatively or additionally thereto, the first wall section of the bottom part may define an entire bottom portion of the bottom part. In a particular preferred embodiment, the pharmaceutical carrier consists of the lid part and the bottom part, i.e. is designed in the form of a two-piece component without any additional elements. Further preferred embodiments are described herein below, and in the claims.

### BRIEF DESCRIPTION OF THE DRAWINGS

FIG. 1 shows various designs of a pharmaceutical carrier.

FIG. 2 shows sectional views of a lid part and a bottom part of an exemplary embodiment of the pharmaceutical carrier according to FIG. 1 including detailed views of a closing mechanism provided on the lid part and the bottom part.

FIG. 3A shows a three-dimensional view of the carrier bottom part as shown on the right in FIG. 2.

FIG. 3B shows a further detailed view of the closing mechanism provided on the lid part and the bottom part of the pharmaceutical carrier according to FIG. 2.

FIG. 4 shows the stability study for carriers filled with API-1 in (A) the closed state and (B) the open state, after storage for twelve weeks at 50° C., 75% RH. The study each compares a control of API-1 alone (uppermost graph with open circles) with the PVOH carriers prepared from the PVOH formulation of the present disclosure (the graph with the filled circles), and the PEO carriers (1) (filled diamonds), (2) (filled squares), or (3) (filled triangles).

FIG. 5 shows the stability study for carriers filled with API-1 in (A) the open state, and (B) the close state. The study each compares the controls of API-1 alone (dashed and solid lines the with each open circles), the PVOH carriers prepared from formulation (1) (solid line, filled circles), the PEO carriers (dashed line, open squares) and the PVOH carriers prepared from the historic formulation not comprising the disintegration aid (dashed line, filled circles).

FIG. 6 shows the stability study for carriers filled with API-2 in (A) the closed state and (B) the open state, after storage for twelve weeks at 50° C., 75% RH. The study each compares a control of API-2 alone (uppermost graph with open circles) with the PVOH carriers prepared from the PVOH formulation of the present disclosure (the graph with the filled circles), and the PEO carriers (1) (filled diamonds), (2) (filled squares), or (3) (filled triangles).

### DETAILED DESCRIPTION OF THE INVENTION

Commercially available capsules are manufactured via a dip coating process. This involves having a reservoir of polymer/water mix and dipping in pins such that they become coated with the mix. The pins are then lifted out of the mix, and the polymer mix on the pin is dried to form a hard capsule before being removed. Prescido™ carriers on the other hand, are manufactured via injection moulding. Injection moulding involves melting of materials in a screw which is then used to inject the melt at high pressure into a mould where it is rapidly cooled before being ejected. This process has a number of advantages over dip coating: the process can be extremely precise, as electric drivers precisely control movement of the machine, which together with very tight control of process parameters such as temperature and pressure and precision mould manufacture, results in high uniformity of parts.

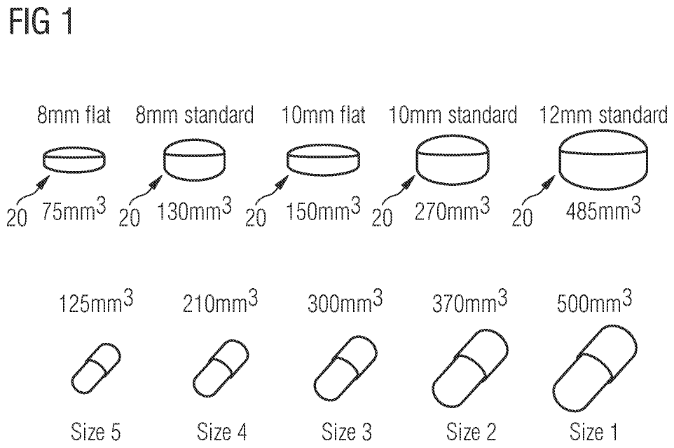

Prescido™ containers are capsules that are filled similar to a capsule, but have appearance of a film-coated tablet. This creates additional presentation options for marketing to choose from in case a dosage form presentation other than a conventional capsule is desired. FIG. 1 (top row) shows a range of designs of the Prescido™ platform.

As becomes apparent from FIG. 1, the Prescido™ containers may have different designs and different filling volumes. Specifically, the containers may have various diameters and heights so that an appropriate container may be chosen, for example depending on the volume of powder to be filled into the containers.

In addition, the use of injection moulding opens up opportunities for complicated part geometries. In dip moulding, both the outer and inner geometries of the capsule are limited to the shape of the pins whereas the shape of injection moulded parts is defined by the mould shape, which can allow multiple features on each face of the carrier.

The composition of traditional capsules is limited to polymers which have suitable rheological and film forming properties when dispersed in water. Injection moulding however, is a hot melt process, which necessitates very different material properties. This presents both an opportunity to move away from traditional capsule materials such as gelatin (animal derived, mechanical properties dependent on environmental conditions) and HPMC (dissolution lag time) and a challenge as the injection moulding process is very demanding with respect to required material properties. The materials must be thermally stable during the process, have good melt flow properties—particularly under high shear conditions, be flexible enough when cooled to be ejected from the machine and for this application be mechanically strong to enable pharmaceutical processing and dissolve quickly in water. In addition the material must be suitable for human consumption and be approved for pharmaceutical use.

The present inventors have found that a formulation suitable for injection moulding can be based on polyvinyl alcohol (PVOH), see Example 1 herein below. Different contents of PVOH were tested to achieve a formulation with the desired physico-chemical properties.

Accordingly, the present disclosure provides a formulation for injection moulding of a pharmaceutical carrier, wherein the formulation comprises 27-85% (w/w) of polyvinyl alcohol, and 10-60% (w/w) of a disintegration aid selected from maize starch, wheat starch, and combinations thereof; and optionally one or more excipients. Preferably, the disintegration aid is maize starch.

Suitable formulations for injection moulding of a pharmaceutical carrier comprise 27-85% (w/w) polyvinyl alcohol. Amounts of less than 27% (w/w) of polyvinyl alcohol results in too weak mechanical properties, such that the pharmaceutical carrier produced from the formulation of the present invention is expected to not have adequate closure force. Accordingly, in embodiments, the formulation comprises 35-82% (w/w) polyvinyl alcohol, preferably 40-80% (w/w), more preferably 45-75% (w/w), more preferably 50-70% (w/w), more preferably 55-68% (w/w), more preferably 60-65% (w/w), and most preferably about 62% (w/w) of said polyvinyl alcohol.

A particular suitable polyvinyl alcohol is polyvinyl alcohol (4-88), which combines a good solubility with a good processability during injection moulding. Polyvinyl alcohol with higher molecular weights have a less desirable dissolution rate, and are difficult to process. The relationship between an increase of the molecular weight polymer and a decrease in dissolution rate has been studied previously (see for example, Ueberreiter K. The solution process. In: Crank J, Park G S, editors. Diffusion in polymers. New York, N.Y.: Academic Press; 1968. p. 219-57; Miller-Chou, B and Koenig, J., A review of polymer dissolution, Prog. Polym. Sci. 2003, 28: 1223-1270). In addition, a degree of hydrolyzation of about 88% provides good dissolution properties in vivo. A significantly higher degree of hydrolyzation results in a decrease of the dissolution rate, and/or requires higher temperatures for good dissolution.

However, in order to provide suitable properties in terms of dissolution rate and rigidity, it is necessary to incorporate a suitable amount of a disintegration aid (or pore former), while maintaining a good processability by injection moulding. As shown in the examples, several disintegration aids (pore formers) were tested, resulting in less favorable mechanical properties, a poorer dissolution or slight lag time in dissolution, phase separation during stability studies, or required too high pressures during injection moulding. Surprisingly, it was found that mixtures of polyvinyl alcohol with maize starch or wheat starch provided good mechanical properties and dissolution rate, while still being well processable by injection moulding. Among the two starches, maize starch performed slightly better than wheat starch. Thus, in a preferred embodiment, the disintegration aid is maize starch.

Upon further testing, it was found that suitable formulations for injection moulding of a pharmaceutical carrier comprise 10-60% (w/w) of said disintegration aid. Accordingly, formulations for injection moulding of a pharmaceutical carrier comprise, depending on the content of PVOH, 10-60% (w/w) of maize starch, wheat starch, or combinations thereof. Amounts of less than 10% (w/w) of the disintegration aid results in lag time during dissolution. Amounts of more than 60% (w/w) of said disintegration aid results in a too weak mechanical properties, such that the pharmaceutical carrier produced from the formulation of the present invention is expected to not have adequate closure force. Particularly suitable formulations for injection moulding of a pharmaceutical carrier of the present disclosure may comprise 15-55% (w/w), preferably 17.5-50% (w/w), preferably 20-45% (w/w), preferably 22.5-40% (w/w), preferably 25-37.5% (w/w), preferably 27.5-35% (w/w), even more preferably 28-32% (w/w), and most preferably about 30% of said disintegration aid.

Mechanical properties can be assessed via two methods, determining the puncture force and the ‘snap open’ force. Firstly, puncture force is measured by a standard texture analyzer equipped with a flat faced pin of defined surface area, which exerts pressure on the carrier part until the material fails and is punctured. From the pressure and the pin surface area the force is calculated. Additionally, mechanically strong formulations are also be measured for ‘snap-open’ force. In these measurements empty closed carriers are placed in a standard tablet crusher, and the force is measured at which the carriers snap open.

The dissolution rate of a capsule can be determined using the ‘assay for immediate release’ as described in the US Pharmacopeia, section <711> from 2011. The assay uses the USP apparatus I (basket) and Fasted state simulating gastric fluid (FasSGF; commercially available) at 37° C. and 100 rpm with n=3 capsules. In one embodiment, the pharmaceutical carrier exhibits a dissolution rate of at least 80%, preferably at least 85%, more preferably at least 90%, and most preferably at least 95% drug substance within 15 minutes; using a fast dissolving compound, e.g. propanolol.HCl as the test substance. A fast dissolving compound is, for example, a BCS (Biopharmaceutical Classification System) Class 1 or Class 3 compound.

The formulation may also comprises an excipient. The excipient may be at least one selected from the list consisting of lubricant, process aid, colorant, opacifier, filler, and glidant.

Usually, the formulation will further comprise a lubricant, which helps to release the moulded carrier from the mould, and generally reduces stickiness. For example, the formulation may comprise 0.3-3.0% (w/w) of a lubricant. In amounts above 3% (w/w), it is expected that the lubricant will impact the dissolution rate. On the other hand, at least 0.3% (w/w) of lubricant is required in order to render the pressure during the injection moulding step sufficiently low. In further embodiments, the formulation may preferably comprise 0.5-2.8% (w/w), more preferably 1.0-2.6% (w/w), more preferably 1.2-2.6% (w/w), more preferably 1.4-2.4% (w/w), more preferably 1.6-2.2% (w/w), more preferably 1.8-2.1% (w/w), and most preferably about 2% (w/w) of a lubricant. The lubricant may be stearic acid or one of its salts such as magnesium stearate or calcium stearate, sodium stearyl fumarate (SSF), stearyl alcohol, hydrogenated vegetable oil, glyceryl behenate, or any combination thereof. In preferred embodiments, the lubricant is stearic acid or one of its salts such as magnesium stearate or calcium stearate. A particularly suitable lubricant is stearic acid.

Moreover, depending on the amount of polyvinyl alcohol, it may be advantageous to further incorporate a process aid into the formulation of the present disclosure. The process aid improves the flowability and allows reducing the temperature and pressure in the injection moulding process. While 5% (w/w) of process aid are optimal for the mechanical properties of the pharmaceutical carrier, also higher amounts may be used. However, above 14%, the formulation becomes too soft, and the mechanical properties are less favorable. As a consequence, the carrier opening force is expected to be problematic. At the same time, the dissolution rate starts to decrease with increasing amounts of process aid. Accordingly, the formulation may comprise 5-14% (w/w) of a process aid, preferably 5-12% (w/w), more preferably 5-10% (w/w), more preferably 5-8% (w/w), even more preferably 5-6% (w/w), and most preferably about 5% (w/w) of a process aid. A particular suitable process aid for use in the formulation of the present disclosure is propan-2-glycol.

It is further contemplated that the formulation of the present disclosure comprises a colorant and/or an opacifier. Colorants and opacifiers are added for aesthetic reasons only, and are not required or critical for the formulation. The amount of colorant and/or opacifier may be adapted to the respective use, but is technically not limited to a specific amount. For example, the formulation may comprises 0-6% (w/w) of one or more colorant and/or opacifier, preferably 0.01-5% (w/w) of one or more colorant and/or opacifier, more preferably 0.25-4% (w/w) of one or more colorant and/or opacifier, more preferably 0.5-3% (w/w) of one or more colorant and/or opacifier, more preferably 0.75-2.5% (w/w) of one or more colorant and/or opacifier, more preferably 1-2% (w/w) of one or more colorant and/or opacifier, more preferably 1-1.5% (w/w) of one or more colorant and/or opacifier, and most preferably about 1% (w/w) of one or more colorant and/or opacifier. The colorant and/or opacifier may be any suitable known in the art. For example, the colorant and/or opacifier may be selected from titanium dioxide, iron oxide, lake pigments, mica-based pigments (e.g., Candurin), formulated pigments (e.g., Opadry®), and any combination thereof.

The formulation may optionally comprise a glidant, in order to improve the flow of blend before injection moulding. If present, the glidant may be, for example, colloidal silicon dioxide.

Alternative excipients and examples of colorant, opacifier, glidant, lubricant, will be apparent to the skilled person, and described in well-known reference books such as Remington, Handbook of Pharmaceutical Excipients.

The components and amounts thereof are selected to result in an injection pressure of 1112-2760 bar, preferably 1200-2750 bar, more preferably 1300-2740 bar, more preferably 1400-2730 bar, more preferably 1500-2720 bar, more preferably 1600-2710 bar, in particular 1700-2700 bar, using a Demag IntElect 50-45 machine for injection moulding. The skilled person would be able to adapt the injection pressure ranges to different injection moulding machines and to account for variations in melt temperature. The injection pressure has to be balanced with gate vestige and is geometry and formulation dependent. In addition, the components and amounts thereof are selected to comply with a bulk temperature of 160° C.-220° C., preferably 160° C.-210° C., more preferably 160° C.-200° C., more preferably 170° C.-200° C., more preferably 180-200° C., in particular 185° C.-195° C., such as about 190° C. Increasing bulk temperatures require optimization of the residence times. Usually mould temperatures are selected to be about 25-50° C., e.g. 30-40° C.

One particularly suitable embodiment of the formulation is shown in the examples. In this embodiment, the formulation comprises 60-65% (w/w) polyvinyl alcohol (4-88), 28-32% (w/w) of maize starch, 1.8-2.1% (w/w) of stearic acid, and 5-6% (w/w) of propan-2-glycol.

As shown in the examples, the pharmaceutical carriers produced by injection moulding using the formulation of the present disclosure surprisingly maintain a high stability of the active pharmaceutical ingredient (API), when tested under stressed conditions such as storage at 50° C. Thus, it is particular advantageous that at least the same and/or improved stability is observed when using the formulation of the present disclosure as compared to comparative examples.

The present disclosure further provides a method of producing a pharmaceutical carrier, comprising the steps of (a) melting a formulation as described above, and (b) injecting the melt into a mould. Said method may optionally comprise a further step (c) cooling the injected melt and optionally ejecting the moulded material. As described above, preferably the pharmaceutical carrier is a capsule, and at least one lid part and at least one bottom part is formed.

At least one of the lid part and the bottom part has a first wall section with a thickness of 180-250 μm, preferably 185-225 μm, and even more preferably 190-220 μm, and a second wall section with a thickness of 350-450 μm, preferably 375-425 μm, more preferably 390-410 μm, and most preferably about 400 μm. In a preferred embodiment the first wall section (26) of the lid part (22) defines an entire top portion of the lid part (22) and/or wherein the first wall section (30) of the bottom part (24) defines an entire bottom portion of the bottom part (24).

In a particular preferred embodiment, the pharmaceutical carrier consists of the lid part and the bottom part, i.e. is designed in the form of a two-piece component without any additional elements.

Preferably, the pharmaceutical carrier is tablet shaped, i.e. designed to have the functionality of a standard pharmaceutical capsule while maintaining the patient appeal of a tablet. In particular, the containers are typically selected to have a tablet shape, such as a disc shape, as opposed to a capsule shape. When considering the lid and bottom part of the pharmaceutical carrier, a capsule shape would be elongated along a central axis running from a center of the bottom part to a center of the lid part. Thus for a traditional capsule, a ratio of a lateral extension, in particular a diameter of the lid and bottom part to a height of the assembled lid and bottom parts along the central axis would be less than 1:1, such as 0.5:1 or less. For example a size 0 capsule has a diameter of 7.64 mm and a height of 21.7 mm (ratio of 0.35:1) and a size 3 capsule has a diameter of 5.82 mm and a height of 15.9 mm (also a ratio of 0.37:1). In contrast a tablet-shaped carrier has a flatter shape and would have a ratio of greater than 1 (1:1 being essentially a sphere). Thus, the pharmaceutical carrier preferably is designed such that the ratio of a lateral extension, in particular a diameter of the lid and bottom part to the height of the assembled lid and bottom parts is >1, preferably ≥1.4, more preferably ≥1.5, even more preferably ≥2, most preferably ≥2.4 and in particular ≥2.5. The containers depicted in FIG. 1, from left to right, have a ratio of a lateral extension of the lid part and the bottom part to a height of the assembled lid and bottom parts of 1:0.4, i.e. of 2.5, 1:0.7, i.e. of 1.43, 1:0.42, i.e. of 2.38, 1:0.875, i.e. 1.14, 1:0.69, i.e. of 1.45.

The thickness of the first wall section has been optimized at 190 to 220 μm. This is thick enough such that, during manufacturing of the pharmaceutical carrier via injection moulding, the material can flow through the thin first wall section, and still reliably fill the thicker walled area of the second wall section while being thin enough to achieve the rapid carrier disintegration required to achieve immediate release dissolution profiles of filled compounds. The second wall section has been optimized to a thickness of 400 μm. Here the balance is between having a greater internal volume available for filling, and having the mechanical strength required for filling and handling (including resistance to opening once filled).

A first wall section of the lid part may define at least a portion of a top portion of the lid part. Preferably, the first wall section of the lid part defines the entire top portion of the lid part such that, upon disintegration of the thin first wall section, a rapid and reliable release of compounds filled into the pharmaceutical carrier via the disintegrating top portion of the lid part is achieved.

A second wall section of the lid part may define at least a portion of a side wall portion of the lid part. For example, the second wall section of the lid part may define a shoulder or corner of the lid part which is arranged adjacent to the top portion of the lid part. Specifically, the second wall section of the lid part may extend from the first wall section, i.e. in particular the top portion of the lid part, along an outer circumference thereof, in the direction of the bottom part. This design provides the lid part with the mechanical stability which is required to handle the lid part and to connect it with the bottom part so as to form the pharmaceutical carrier as desired.

In the context of this application, the expression “side wall portion of the lid part” defines a portion of the lid part which extends substantially parallel to the central axis of the pharmaceutical carrier. Preferably, the side wall portion of the lid part has a circular cylindrical shape and surrounds the central axis of the pharmaceutical carrier substantially parallel therewith. The expression “top portion of the lid part” defines a portion of the lid part which is connected to the side wall portion and “covers” a free space surrounded by side wall portion at one end thereof. The top portion of the lid part might extend substantially perpendicular with respect to the central axis of the pharmaceutical carrier, wherein, however, in a particular preferred embodiment, the top portion is at least slightly curved with respect to the central axis of the pharmaceutical carrier. When viewed from “outside” of the carrier, the top portion of the lid part in particular is provided with a concave curvature.

In a preferred embodiment of the pharmaceutical carrier, a first wall section of the bottom part defines at least a portion of a bottom portion of the bottom part. Preferably, the first wall section of the bottom part defines the entire bottom portion of the bottom part such that, upon disintegration of the thin first wall section, a rapid and reliable release of compounds filled into the pharmaceutical carrier via the disintegrating bottom portion of the bottom part is achieved.

A second wall section of the bottom part may define at least a portion of a side wall portion of the bottom part. Specifically, the second wall section of the bottom part may extend from the first wall section, i.e. in particular the bottom portion of the bottom part, along an outer circumference thereof, in the direction of the lid part. Preferably, the height of the second wall section of the bottom part is larger than the height of the second wall section of the lid part. In other words, in a preferred embodiment of the pharmaceutical carrier, the bottom part has a generally hollow cylindrical shape and hence defines a “vessel” which may be filled with the pharmaceutical compound. To the contrary, the lid part, which may be provided with a second wall section which merely defines a shoulder or corner surrounding the top portion of the lid part, may have a generally “flat” shape. The larger wall thickness of the second wall section as compared to the first wall section provides the bottom part with a mechanical strength and stability which allows an unhindered filling of the bottom part with the pharmaceutical compound.

In the context of this application, the expression “side wall portion of the bottom part” defines a portion of the bottom part which extends substantially parallel to the central axis of the pharmaceutical carrier. Preferably, the side wall portion of the bottom part has a circular cylindrical shape and surrounds the central axis of the pharmaceutical carrier substantially parallel therewith. The expression “bottom portion of the bottom part” defines a portion of the bottom part which is connected to the side wall portion and “covers” a free space surrounded by side wall portion at one end thereof. The bottom portion of the lid part might extend substantially perpendicular with respect to the central axis of the pharmaceutical carrier, wherein, however, in a particular preferred embodiment, the bottom portion is at least slightly curved with respect to the central axis of the pharmaceutical carrier. When viewed from “outside” of the carrier, the bottom portion of the bottom part in particular is provided with a concave curvature.

In preferred embodiments, the lid part and the bottom part are connected to each other by a complementary closing mechanism. The complementary closing mechanism provides for a reliable and easy to establish connection between the lid part and the bottom part.

More specifically, the closing mechanism may comprise a first snap part which projects from the second wall section of the bottom part so as to face and to interact with a second snap part which projects from the second wall section of the lid part. Upon closing the pharmaceutical carrier, i.e. upon connecting the lid part to the bottom part, at least one of the first and the second snap part may be elastically deformed. When the lid part and the bottom part have reached their final relative positions, i.e. when the lid part is positioned on top of the bottom part so as to seal the interior of the bottom part as desired, the elastic information of the at least one of the first and the second snap part may be released in such a manner that the snap parts intact with each other so as to reliably connect the lid part and the bottom part.

For example, the first snap part may comprise a projection which is adapted to engage with a corresponding projection provided on the second snap part so as to counteract separation of the first snap part and the second snap part and thus separation of the lid part and the bottom part. In particular, the projection of the first snap part may comprise a first abutting surface which faces the bottom part and which is adapted to abut against a second abutting surface which is formed on the second snap part and which faces the lid part when the bottom part and the lid part are connected to each other. The first abutting surface formed on the first snap part may extend at an angle of 90 to 150° relative to the side wall portion of the bottom part. The second abutting surface formed on the second snap part may extend at an angle of 90 to 150° relative to the side wall portion of the lid part.

The projection provided on the first snap part may taper in a direction of a free end of the first snap part so as to form a first inclined engagement surface. The first inclined engagement surface may be adapted to engage with a second inclined engagement surface formed on the projection provided on the second snap part which tapers in a direction of a free end of the second snap part. Upon connecting the lid part to the bottom part of the pharmaceutical carrier, the second inclined engagement surface may slide along the first inclined engagement surface thus guiding the projection provided on the first snap part into engagement with the corresponding projection provided on the second snap part. As a result, connecting the lid part to the bottom part is simplified.

One of the first and the second snap part may project from the second wall section of the lid part or the bottom part in the region of an inner circumference of the second wall section, wherein the other one of the first and the second snap part may project from the second wall section of the lid part or the bottom part in the region of an outer circumference of the second wall section of the bottom part. Preferably, the first snap part provided on the bottom part of the pharmaceutical carrier extends from the second wall section of the bottom part in the region of an inner circumference of the second wall section. A thus designed first snap part is particularly suitable for interaction with a second snap part which projects from a particularly shoulder- or corner-shaped second wall section of the lid part in the region of an outer circumference of the second wall section of the lid part.

The closing mechanism may further comprise an inner rib which projects from the second wall section of the lid part or the bottom part in the region of an inner circumference of the second wall section at a distance from the first or the second snap part which projects from the second wall section of the lid part or the bottom part in the region of an outer circumference of the second wall section. In particular, the closing mechanism may comprise inner rib which projects from the second wall section of the lid part in the region of an inner circumference thereof and hence at a distance from the second snap part which projects from the particularly shoulder- or corner-shaped second wall section of the lid part in the region of an outer circumference thereof. As a result, the inner rib and the second snap part define a gap therebetween which is adapted to accommodate the first snap part when the lid part and the bottom part of the pharmaceutical carrier are connected to each other. In the connected state of the lid part and the bottom part, the first snap part is held in place in the gap between the inner rib and the second snap part due to the interaction with the second snap part, i.e. in particular you to the interaction of the first abutting surface formed on the first snap part with the second abutting surface formed on the second snap part, while the inner rib provides for additional mechanical stability and stiffness of the closing mechanism.

It is, however, also conceivable to provide the bottom part of the pharmaceutical carrier with an inner rib, in particular in case the bottom part is provided with a first snap part which projects from the second wall section of the bottom part in the region of an outer circumference thereof and which is adapted to interact with a second snap part which projects from the second wall section of the lid part in the region of an inner circumference thereof. In this case, the inner rib and the first snap part may define a gap therebetween which is adapted to accommodate the second snap part when the lid part and the bottom part of the pharmaceutical carrier are connected to each other.

Preferably, the inner rib is shorter than the snap part arranged opposite to the inner rib. In other words, preferably, the snap part which, together with the inner rib, defines a gap for accommodating the other snap part projects further from the second wall section of the lid part or the bottom part than the inner rib. Further, the inner rib may taper in a direction of a free end of the inner rib so as to form a third inclined engagement surface facing the first or the second snap part which projects from the second wall section of the lid part or the bottom part in the region of an outer circumference of the second wall section and hence is arranged opposite to the inner rib. Preferably, the third inclined engagement surface provided on the inner rib extends substantially parallel to the abutting surface provided on the projection of the snap part arranged opposite to the inner rib. As a result, the snap part which is adapted to be accommodated in the gap defined between the inner rib and the snap part arranged opposite to the inner rib upon connecting the lid part and the bottom part of the pharmaceutical carrier is guided into engagement with the snap part arranged opposite to the inner rib.

In a preferred embodiment of the pharmaceutical carrier, the first wall section of the lid part, in particular in a region which is defined by a material injection point into a mould upon manufacturing of the lid part, is provided with a depression. This depression may have a wall thickness that is larger than the wall thickness of the remaining part of the first wall section, but smaller than the wall thickness of the second wall section of the lid part. For example, the depression may be arranged in a central region of a top portion of the lid part. A sign which indicates a cavity in which the lid part was moulded on a multicavity moulding tool during an injection moulding process may be imprinted onto a surface, in particular an inner surface of the depression. This allows for automatic sorting of the lid parts by cavity for applications where tight weight uniformity is required.

Alternatively or additionally thereto, the first wall section of the bottom part, in particular in a region which is defined by a material injection point into a mould upon manufacturing of the bottom part, is provided with a depression. This depression may have a wall thickness that is larger than the wall thickness of the remaining part of the first wall section, but smaller than the wall thickness of the second wall section of the lid part. For example, the depression may be arranged in a central region of a bottom portion of the bottom part. A sign which indicates a cavity in which the bottom part was moulded on a multicavity moulding tool during an injection moulding process may be imprinted onto a surface, in particular an inner surface of the depression. This allows for automatic sorting of the bottom parts by cavity for applications where tight weight uniformity is required.

At least one of the lid part and the bottom part, in the region of an inner surface thereof, may be provided with a plurality of inner protrusions which project radially inwards from an inner surface of the second wall section and/or an inner surface of the inner rib. In case the lid part or the bottom part which is provided with inner protrusions also is provided with an inner rib, the inner protrusions, in a direction of a central axis of the lid part or the bottom part, may extend from the top portion of the lid part or the bottom portion of the bottom part along the second wall section of the lid part of the bottom part and finally along the inner rib which projects from the second wall section in the region of an inner circumference thereof. In case the lid part of the bottom part which is provided with inner protrusions does not comprise an inner rib, the inner protrusions, in a direction of a central axis of the lid part or the bottom part, may extend from the top portion of the lid part or the bottom portion of the bottom part along the second wall section of the lid part or the bottom part. At least one of and in particular each of the inner protrusions may comprise a projecting nose which projects beyond the second wall section and/or the inner rib.

The inner protrusions, in particular when being provided with projecting noses, reduce a phenomenon termed ‘nesting’, i.e. an adherence of the parts and/or bottom parts stacked on top of each other. As a result, difficulties during manual and automated handling which may be caused by ‘nests’ of stacked parts which are difficult to separate can be eliminated.

The pharmaceutical carrier may be filled with neat API. In this context, the expression “neat API” designates an API comprising at most 5% (w/w) of an additive throughout all development stages of the pharmaceutical drug including its final commercial production. In particular, the neat API within the pharmaceutical carrier may comprise at most 5% (w/w) of an additive, preferably at most 4% (w/w), more preferably at most 3% (w/w), even more preferably at most 2% (w/w), and most preferably at most 1% (w/w).

In a preferred embodiment, a pharmaceutical carrier comprises a lid part and a bottom part. At least one of the lid part and the bottom part has a first wall section with a thickness of 180-250 μm, preferably 185-225 μm, and even more preferably 190-220 μm, and a second wall section with a thickness of 350-450 μm, preferably 375-425 μm, more preferably 390-410 μm, and most preferably about 400 μm. The first wall section of the lid part defines an entire top portion of the lid part. Alternatively or additionally thereto, the first wall section of the bottom part defines an entire bottom portion of the bottom part.

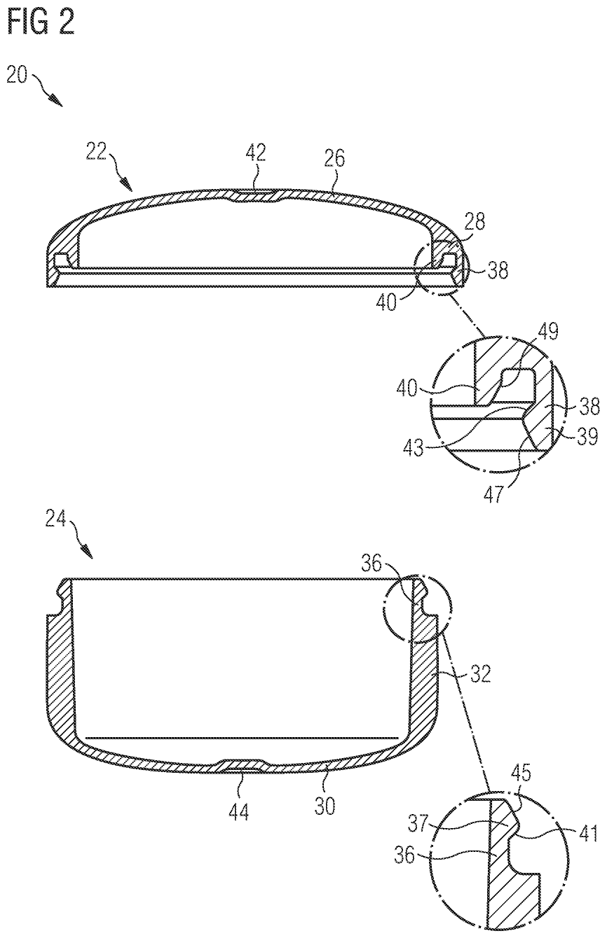

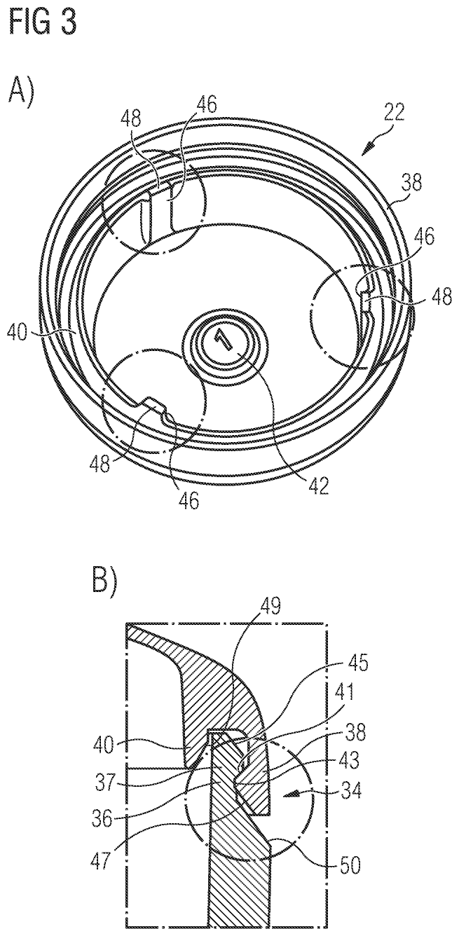

An exemplary pharmaceutical carrier 20 as depicted in FIG. 1 is shown in greater detail in FIGS. 2, 3A and 3B. The carrier 20 comprises a lid part 22 and a bottom part 24. Specifically, the carrier 20 is designed of a two-part component and consists of the lid part 22 and the bottom part 24. The lid part 22, which is shown on the left in FIG. 2 and in FIG. 3A, comprises a first wall section 26 which defines a top portion of the lid part 22 and a second wall section 28 which defines a side wall portion of the lid part 22. In particular, the second wall section 28 of the lid part 22 defines a shoulder or corner of the lid part 22 which is arranged adjacent to the top portion of the lid part 22. Specifically, the second wall section 28 of the lid part 22 extends from the top portion of the lid part 22, along an outer circumference thereof, in the direction of the bottom part 24. The first wall section 26 has a wall thickness that is smaller than a wall thickness of the second wall section 28. In the preferred embodiment of the carrier 20 shown in FIG. 2, the first wall section 26 has a wall thickness of 190 to 220 μm, whereas the second wall section 28 has a wall thickness of about 400 μm.

Similarly, the bottom part 24, which is shown on the right in FIG. 2, comprises a first wall section 30 which defines a bottom portion of the bottom part 24 and a second wall section 32 which defines a side wall portion of the bottom part 24. The second wall section 32 of the bottom part 24 extends from the bottom portion of the bottom part 24 along an outer circumference thereof in the direction of the lid part 22. The first wall section 30 has a wall thickness that is smaller than a wall thickness of the second wall section 32. In the preferred embodiment of the carrier 20 shown in FIG. 2, the first wall section 30 has a wall thickness of 190 to 220 μm, whereas the second wall section 32 has a wall thickness of about 400 μm.

The lid part 22 and the bottom part 24 are connected to each other by means of a complementary closing mechanism 34 which is illustrated in greater detail in the detailed views shown in FIG. 2 as well as in FIG. 3B. The closing mechanism 34 comprises a first hook-shaped snap part 36 which projects from the second wall section 32 of the bottom part 24 in the region of an inner circumference of the second wall section 32. The first hook-shaped snap part 36 faces and interacts with a correspondingly shaped second hook-shaped snap part 38 which projects from the second wall section 28 of the lid part 22 in the region of an outer circumference of the second wall section 28. It would, however, also be conceivable to provide the closing mechanism 34 with a first snap part 36 which projects from the second wall section 32 of the bottom part 24 in the region of an outer circumference of the second wall section 32 and a second snap part 36 which projects from the second wall section 28 of the lid part 22 in the region of an inner circumference of the second wall section 28.

As becomes apparent from the detailed views shown in FIG. 2 and FIG. 3B, the first snap part 36 comprises a projection 37 which, upon connecting the lid part 22 and the bottom part 24, is adapted to engage with a corresponding projection 39 provided on the second snap part 38. The projection 37 of the first snap part 36 comprises a first abutting surface 41 which faces the bottom part 24. Similarly, the projection 39 of the lid part 22 comprises a second abutting surface 43 which faces the lid part 22. The first abutting surface 41 formed on the projection 37 of the first snap part 36 extends at an angle of approximately 135° relative to the side wall portion of the bottom part 24. The second abutting surface 43 formed on the projection 39 of the second snap part 38 extends at an angle of approximately 135° relative to the side wall portion of the lid part 22. Further, the projection 37 provided on the first snap part 36 tapers in a direction of a free end of the first snap part 36 so as to form a first inclined engagement surface 45. Similarly, the projection 39 provided on the second snap part 38 also tapers in a direction of a free end of the first snap part 38 so as to form a second inclined engagement surface 47.

The closing mechanism 34 further comprises an inner rib 40 which projects from the shoulder- or corner-shaped second wall section 28 of the lid part 22 in the region of an inner circumference of the second wall section 28. Hence, the inner rib 40 projects from the second wall section 28 of the lid part 22 at a distance from the second snap part 36 which projects from the second wall section 28 of the lid part 22 in the region of an outer circumference of the second wall section 28. As a result, the inner rib 40 and the second snap part 38 define a gap therebetween which is adapted to accommodate the first snap part 36 when the lid part 22 and the bottom part 24 of the pharmaceutical carrier 20 are connected to each other. However, in case the lid part 22 is provided with a second snap part 38 which is arranged in the region of an inner circumference of the second wall section 28 so as to interact with a first snap part 38 which is arranged in the region of outer circumference of the second wall section 32 of the bottom part 24, it is also conceivable that the closing mechanism 34 comprises an inner rib 40 which projects from the second wall section 32 of the bottom part 24 in the region of an inner circumference of the second wall section 32. In this case it is the first snap part 36 which, together with the inner rib 40, defines a gap which is adapted to accommodate the second snap part 38 when the lid part 22 and the bottom part 24 of the pharmaceutical carrier 20 are connected to each other.

The inner rib 40 is shorter than the second snap part 38 arranged opposite to the inner rib 40, i.e. the second snap part 38 projects further from the second wall section 28 of the lid part 22 than the inner rib 40. Further, the inner rib 40 tapers in a direction of a free end of the inner rib 40 so as to form a third inclined engagement surface 49 facing the second snap part 38 which projects from the second wall section 28 of the lid part 22 in the region of an outer circumference of the second wall section 28 and opposite to the inner rib 40. The third inclined engagement surface 49 extends substantially parallel to the second abutting surface 43 provided on the projection 39 of the second snap part 38 arranged opposite to the inner rib 40. In case the lid part 22 is provided with a second snap part 38 which is arranged in the region of an inner circumference of the second wall section 28 so as to interact with a first snap part 38 which is arranged in the region of outer circumference of the second wall section 32 of the bottom part 24, the third inclined engagement surface 49 formed on the inner rib 40 may face the first snap part 36 which projects from the second wall section 32 of the bottom part 24 in the region of an outer circumference of the second wall section 32 and opposite to the inner rib 40

Upon closing the pharmaceutical carrier 20, i.e. upon connecting the lid part 22 to the bottom part 24, the first inclined engagement surface 45 provided on the projection 37 of the first snap part 36 comes into contact with the second inclined engagement surface 47 provided on the projection 39 of the second snap part 38. When the lid part 22 approaches the bottom part 24, the second inclined engagement surface 47 slides along the first inclined engagement surface 45 which results in a slight elastic deformation of the first and the second snap part 36, 38. Specifically, the first snap part 38 is slightly bent radially inwards, whereas the second snap part 36 is slightly bent radially outwards. Inward bending of the first snap part 38 is, however, limited by the inner rib 40. Further, the third inclined engagement surface 49 provided on the inner rib 40 guides the second snap part 38 into its final position in the gap defined between the second snap part 38 and the inner rib 40, see FIG. 3B.

When the lid part 22 and the bottom part 24 have reached their final relative positions, i.e. when the lid part 22 is positioned on top of the bottom part 24 so as to seal the interior of the bottom part 24, the elastic deformation of the first and the second snap part 36, 38 is released and the first abutting surface 41 provided on the projection 37 of the first snap part 36 abuts against the second abutting surface 43 provided on the projection 39 of the second snap part 38. The interaction between the first and the second abutting surface 41, 43 contacts separation of the bottom part 24 and the lid part 22. The inner rib 40 provides for additional mechanical stability and stiffness of the closing mechanism 34.

The first wall section 26 of the lid part 22, in a central region which is defined by a material injection point into a mould upon manufacturing of the lid part 22, is provided with a depression 42 which has a wall thickness that is larger than the wall thickness of the remaining part of the first wall section 26, but still smaller than the wall thickness of the second wall section 28 of the lid part 22. A number, in the drawings the number “1”, is imprinted onto an inner surface of the depression 42 which indicates a cavity in which the lid part 22 was moulded on a multicavity moulding tool. Similarly, also the first wall section 30 of the bottom part 24, in a central region which is defined by a material injection point into a mould upon manufacturing of the bottom part 24, is provided with a depression 44 which has a wall thickness that is larger than the wall thickness of the remaining part of the first wall section 30, but still smaller than the wall thickness of the second wall section 32 of the bottom part 24. A number (not shown in the drawings) is imprinted onto an inner surface of the depression 44 which indicates a cavity in which the bottom part 24 was moulded on a multicavity moulding tool.

As becomes apparent from FIG. 3A, the lid part 22 further is provided with a plurality of inner protrusions 46 which project radially inwards from an inner surface of the second wall section 28 and an inner surface of the inner ring 40, respectively. In the specific embodiment of a lid part 22 shown in the drawings, three inner protrusions 46 are provided. It is, however, also conceivable to provide the lid part 22 with less than or more than three inner protrusions 46. The inner protrusions 46 serve to prevent jamming of parts 22, which are stacked on top of each other during handling. Each of the inner protrusions 46 comprises a nose 48 which projects from the inner rib 40 and which further reduces the risk of jamming of parts 22 stacked on top of each other. In the embodiment of the carrier 20 which is illustrated in the drawings, only the lid part 22 is provided with inner protrusions 46. It is, however, also conceivable that alternatively or additionally also the bottom part 24 of the carrier 20 is provided with inner protrusions as described herein.

Finally, as becomes apparent from FIG. 3B, the bottom part 24 is provided with an angled balcony 50 which is formed in the region of an outer surface of the second wall section 32 adjacent to the first hook-shaped snap part 36 and which is inclined radially outwards from an outer circumference of the hook-shaped snap part 38 towards an outer surface of second wall section 32. Powder which inadvertently falls onto the balcony 50 upon closing the carrier 20 can easily be removed.

Advantageously, the pharmaceutical carrier exhibits a standard mass deviation of the respective carrier parts of less than 1 mg, preferably less than 0.8 mg, more preferably less than 0.6 mg, even more preferably less than 0.4 mg, still more preferably less than 0.3 mg, still even more preferably less than 0.2 mg, and most preferably less than 0.1 mg, as shown in the examples section herein below.

The invention is further described by the following embodiments.
1. A formulation for injection moulding of a pharmaceutical carrier, wherein the formulation comprises 27-85% (w/w) of polyvinyl alcohol; and 10-60% (w/w) of a disintegration aid selected from maize starch, wheat starch, and combinations thereof; and optionally one or more excipients.
2. The formulation of embodiment 1, wherein said polyvinyl alcohol is polyvinyl alcohol (4-88).
3. The formulation of embodiment 1 or 2, wherein the formulation comprises 35-82% (w/w) polyvinyl alcohol, preferably 40-80% (w/w), more preferably 45-75% (w/w), more preferably 50-70% (w/w), more preferably 55-68% (w/w), more preferably 60-65% (w/w), and most preferably about 62% (w/w) of said polyvinyl alcohol.
4. The formulation of any one of embodiments 1 to 3, wherein the formulation comprises 15-55% (w/w), preferably 17.5-50% (w/w), preferably 20-45% (w/w), preferably 22.5-40% (w/w), preferably 25-37.5% (w/w), preferably 27.5-35% (w/w), even more preferably 28-32% (w/w), and most preferably about 30% of said disintegration aid.
5. The formulation of any one of embodiments 1-4, wherein the disintegration aid is maize starch.
6. The formulation of any one of embodiments 1 to 5, wherein the excipient is at least one selected from the list consisting of lubricant, process aid, colorant, opacifier, and glidant.
7. The formulation of embodiment 6, wherein the formulation comprises 0.3-3.0% (w/w) of a lubricant, preferably 0.5-2.8% (w/w), more preferably 1.0-2.6% (w/w), more preferably 1.2-2.6% (w/w), more preferably 1.4-2.4% (w/w), more preferably 1.6-2.2% (w/w), more preferably 1.8-2.1% (w/w), and most preferably about 2% (w/w) of a lubricant.
8. The formulation of embodiment 6 or 7, wherein the formulation comprises a lubricant, wherein the lubricant is stearic acid or one of its salts such as magnesium stearate or calcium stearate, sodium stearyl fumarate (SSF), stearyl alcohol, hydrogenated vegetable oil, glyceryl behenate, or any combination thereof; preferably wherein the lubricant is stearic acid or one of its salts such as magnesium stearate or calcium stearate.
9. The formulation of embodiment 6 or 7, wherein the formulation comprises a lubricant, wherein the lubricant stearic acid.
10. The formulation of any one of embodiments 6-9, wherein the formulation comprises 5-14% (w/w) of a process aid, preferably 5-12% (w/w), more preferably 5-10% (w/w), more preferably 5-8% (w/w), even more preferably 5-6% (w/w), and most preferably about 5% (w/w) of a process aid.
11. The formulation of any one of embodiments 6-10, wherein the formulation comprises a process aid, wherein the process aid is propan-2-glycol.
12. The formulation of any one of embodiments 6-11, wherein the formulation comprises a colorant and/or an opacifier, preferably wherein the selected from titanium dioxide, iron oxide, lake pigments, mica-based pigments, formulated pigments, and any combination thereof,
13. The formulation of any one of embodiments 6-12, wherein the formulation comprises a colorant and/or an opacifier in amounts of about 1% (w/w).
14. The formulation of any one of embodiments 6-13 wherein the formulation comprises a glidant, in particular wherein the glidant is colloidal silicon dioxide.
15. The formulation of any one of embodiments 1-14, comprising 60-65% (w/w) polyvinyl alcohol (4-88), 28-32% (w/w) of maize starch, 1.8-2.1% (w/w) of stearic acid, and 5-6% (w/w) of propan-2-glycol.
16. A method of producing a pharmaceutical carrier, comprising the steps of
   (a) melting a formulation according to any one of embodiments 1-15, and
   (b) injecting the melt into a mould.
17. The method of embodiment 16, further comprising the step
   (c) cooling the injected melt and optionally ejecting the moulded material.
18. The method of embodiment 16 or embodiment 17, wherein the pharmaceutical carrier (20) is a capsule, and at least one lid part (22) and at least one bottom part (24) is formed.
19. The method of embodiment 18, wherein at least one of the lid part (22) and the bottom part (24) has a first wall section (26, 30) with a thickness of 180-250 μm, preferably 185-225 μm, and even more preferably 190-220 μm, and a second wall section (28, 32) with a thickness of 350-450 μm, preferably 375-425 μm, more preferably 390-410 μm, and most preferably about 400 μm.
20. The method of embodiment 18, wherein the first wall section (26) of the lid part (22) defines an entire top portion of the lid part (22) and/or wherein the first wall section (30) of the bottom part (24) defines an entire bottom portion of the bottom part (24).
21. The method of any one of embodiments 18-20, wherein the pharmaceutical carrier (20) is designed such that a ratio of a lateral extension of the lid and bottom part (22, 24) to a height of the assembled lid and bottom parts (22, 24) is >1, preferably ≥1.4, more preferably ≥1.5, even more preferably ≥2, most preferably ≥2.4 and in particular ≥2.5.
22. The method of anyone of embodiments 18-21, wherein the lid part (22) and the bottom part (24) are connected to each other by a complementary closing mechanism (34);
   in particular wherein the closing mechanism (34) comprises a first snap part (36) which projects from the second wall section (32) of the bottom part (24) so as to face and to interact with a second snap part (38) which projects from the second wall section (28) of the lid part (22);
   more particularly wherein the first snap part (36) comprises a projection (37) adapted to engage with a corresponding projection (39) provided on the second snap part (38) so as to counteract separation of the first snap part (36) and the second snap part (38) and thus separation of the lid part (22) and the bottom part (24);
   even more particularly wherein the projection (37) provided on the first snap part (36) tapers in a direction of a free end of the first snap part (36) so as to form a first inclined engagement surface (45) adapted to engage with a second inclined engagement surface (47) formed on the projection (39) provided on the second snap part (38) which tapers in a direction of a free end of the second snap part (36);
   most preferably wherein one of the first and the second snap part (36, 38) projects from the second wall section (28, 32) of the lid part (22) or the bottom part (24) in the region of an inner circumference of the second wall section (28, 32), and wherein the other one of the first and the second snap part (36, 38) projects from the second wall section (28, 32) of the lid part (22) or the bottom part (24) in the region of an outer circumference of the second wall section (28, 32).
23. The method of embodiment 22, wherein the closing mechanism (34) further comprises an inner rib (40) which projects from the second wall section (28) of the lid part (22) or the bottom part (24) in the region of an inner circumference of the second wall section (28, 32) at a distance from the first or the second snap part (36, 38) which projects from the second wall section (28, 32) of the lid part (22) or the bottom part (24) in the region of an outer circumference of the second wall section (28, 32);
   in particular wherein the inner rib (40) tapers in a direction of a free end of the inner rib (40) so as to form a third inclined engagement surface (49) facing the first or the second snap part (36, 38) which projects from the second wall section (28, 32) of the lid part (22) or the bottom part (24) in the region of an outer circumference of the second wall section (28, 32).
24. The method of any one of embodiments 18-23, wherein the pharmaceutical carrier (20) is filled with neat API.
25. The method of embodiment 18, wherein at least one of the lid part (22) and the bottom part (24) has a first wall section (26, 30) with a thickness of 180-250 μm, preferably 185-225 μm, and even more preferably 190-220 μm, and a second wall section (28, 32) with a thickness of 350-450 μm, preferably 375-425 μm, more preferably 390-410 μm, and most preferably about 400 μm, wherein the first wall section (26) of the lid part (22) defines an entire top portion of the lid part (22) and/or wherein the first wall section (30) of the bottom part (24) defines an entire bottom portion of the bottom part (24), and wherein the pharmaceutical carrier (20) is designed such that a ratio of a lateral extension of the lid and bottom part (22, 24) to a height of the assembled lid and bottom parts (22, 24) is >1, preferably ≥1.4, more preferably ≥1.5, even more preferably ≥2, most preferably ≥2.4 and in particular ≥2.5
26. The method of any one of embodiments 16-25, further comprising the step (d) sorting the carrier parts by mould cavity.
27. A pharmaceutical carrier produced by the method of any one of embodiments 16-26 using the formulation of any one of embodiments 1-15, comprising
   a lid part (22) and
   a bottom part (24),
   wherein at least one of the lid part (22) and the bottom part (24) has a first wall section (26, 30) with a thickness of 180-250 μm, preferably 185-225 μm, and even more preferably 190-220 μm, and a second wall section (28, 32) with a thickness of 350-450 μm, preferably 375-425 μm, more preferably 390-410 μm, and most preferably about 400 μm.
28. The pharmaceutical carrier of embodiment 27, wherein the pharmaceutical carrier (20) is designed such that a ratio of a lateral extension of the lid and bottom part (22, 24) to a height of the assembled lid and bottom parts (22, 24) is >1, preferably ≥1.4, more preferably ≥1.5, even more preferably ≥2, most preferably ≥2.4 and in particular ≥2.5.
29. The pharmaceutical carrier of embodiment 27 or 28, wherein a first wall section (26) of the lid part (22) defines at least a portion of a top portion of the lid part (22), in particular an entire top portion of the lid part (22).
30. The pharmaceutical carrier of any one of embodiments 27-29, wherein a second wall section (28) of the lid part (22) defines at least a portion of a side wall portion of the lid part (22) which in particular extends from the first wall section (26) of the lid part (22), along an outer circumference thereof, in the direction of the bottom part (24).
31. The pharmaceutical carrier of any one of embodiments 27-30, wherein a first wall section (30) of the bottom part (24) defines at least a portion of a bottom portion of the bottom part (24), in particular an entire bottom portion of the bottom part (24).
32. The pharmaceutical carrier of any one of embodiments 27-31, wherein a second wall section (32) of the bottom part (in 24) defines at least a portion of a side wall portion of the bottom part (24) which in particular extends from the bottom portion of the bottom part (24), along an outer circumference thereof, in the direction of the lid part (22).
33. The pharmaceutical carrier of any one of embodiment 27-32, wherein the lid part (22) and the bottom part (24) are connected to each other by a complementary closing mechanism (34).
34. The pharmaceutical carrier of embodiment 33, wherein the closing mechanism (34) comprises a first snap part (36) which projects from the second wall section (32) of the bottom part (24) so as to face and to interact with a second snap part (38) which projects from the second wall section (28) of the lid part (22).
35. The pharmaceutical carrier of embodiment 34, wherein the first snap part (36) comprises a projection (37) adapted to engage with a corresponding projection (39) provided on the second snap part (38) so as to counteract separation of the first snap part (36) and the second snap part (38) and thus separation of the lid part (22) and the bottom part (24).
36. The pharmaceutical carrier of embodiment 35, wherein the projection (37) provided on the first snap part (36) tapers in a direction of a free end of the first snap part (36) so as to form a first inclined engagement surface (45) adapted to engage with a second inclined engagement surface (47) formed on the projection (39) provided on the second snap part (38) which tapers in a direction of a free end of the second snap part (38).
37. The pharmaceutical carrier of any one of embodiments 34 to 36, wherein one of the first and the second snap part (36, 38) projects from the second wall section (28, 32) of the lid part (22) or the bottom part (24) in the region of an inner circumference of the second wall section (28, 32), and wherein the other one of the first and the second snap part (36, 38) projects from the second wall section (28, 32) of the lid part (22) or the bottom part (24) in the region of an outer circumference of the second wall section (28, 32).
38. The pharmaceutical carrier of any one of embodiments 34 to 37, wherein the closing mechanism (34) further comprises an inner rib (40) which projects from the second wall section (28, 32) of the lid part (22) or the bottom part (24) in the region of an inner circumference of the second wall section (28) at a distance from the first or the second snap part (36, 38) which projects from the second wall section (28, 32) of the lid part (22) or the bottom part (24) in the region of an outer circumference of the second wall section (28, 32).
39. The pharmaceutical carrier of embodiment 38, wherein the inner rib (40) tapers in a direction of a free end of the inner rib (40) so as to form a third inclined engagement surface (49) facing the first or the second snap part (36, 38) which projects from the second wall section (28, 32) of the lid part (22) or the bottom part (24) in the region of an outer circumference of the second wall section (28, 32).
40. The pharmaceutical carrier of any one of embodiments 27 to 39, wherein the first wall section (26) of the lid part (22), in particular in a region which is defined by a material injection point into a mould upon manufacturing of the lid part (22), is provided with a depression (42) which has a wall thickness that is larger than the wall thickness of the remaining part of the first wall section (26), but smaller than the wall thickness of the second wall section (28) of the lid part (22), a sign which indicates a cavity in which the lid part (22) was moulded on a multicavity moulding tool in particular being imprinted onto an inner surface of the depression (42), and/or wherein the first wall section (30) of the bottom part (24), in particular in a region which is defined by a material injection point into a mould upon manufacturing of the bottom part (24), is provided with a depression (44) which has a wall thickness that is larger than the wall thickness of the remaining part of the first wall section (30), but smaller than the wall thickness of the second wall section (32) of the bottom part (24), a sign which indicates a cavity in which the bottom part (24) was moulded on a multicavity moulding tool being imprinted onto an inner surface of the depression (44).
41. The pharmaceutical carrier of any one of embodiments 27 to 40, wherein at least one of the lid part (22) and the bottom part (24), in the region of an inner surface thereof, is provided with a plurality of inner protrusions (46) which project radially inwards from an inner surface of the second wall section (28, 32) and/or an inner surface of the inner rib (40), each of the inner protrusions (46) in particular comprising a projecting nose (48) which projects beyond the second wall section (28, 32) and/or the inner rib (40).
42. The pharmaceutical carrier of any one of embodiments 27-41, exhibiting an absolute standard mass deviation of the respective carrier parts of less than 1 mg, preferably less than 0.8 mg, more preferably less than 0.6 mg, even more preferably less than 0.4 mg, still more preferably less than 0.3 mg, still even more preferably less than 0.2 mg, and most preferably less than 0.1 mg.
43. The pharmaceutical carrier of any one of embodiments 27-42, wherein the pharmaceutical carrier exhibits a dissolution rate of at least 80%, preferably at least 85%, more preferably at least 90%, and most preferably at least 95% drug substance within 15 minutes; when tested using the ‘assay for immediate release’ described in the US Pharmacopeia 2011, section <711> using the USP apparatus I (basket) and Fasted state simulating gastric fluid (FasSGF) at 37° C. and 100 rpm with n=3 capsules, and propanolol.HCl as the test substance.
44. The pharmaceutical carrier of any one of embodiments 27-43, wherein the pharmaceutical carrier is filled with an active pharmaceutical ingredient (API) comprising at most 5% (w/w) of an additive, preferably at most 4% (w/w), more preferably at most 3% (w/w), even more preferably at most 2% (w/w), and most preferably at most 1% (w/w).
45. A pharmaceutical carrier produced by the method of any one of embodiments 16-26 using the formulation of any one of embodiments 1-15, comprising
   a lid part (22) and
   a bottom part (24), wherein at least one of the lid part (22) and the bottom part (24) has a first wall section (26, 30) with a thickness of 180-250 μm, preferably 185-225 μm, and even more preferably 190-220 μm, and a second wall section (28, 32) with a thickness of 350-450 μm, preferably 375-425 μm, more preferably 390-410 μm, and most preferably about 400 μm, wherein the first wall section (26) of the lid part (22) defines an entire top portion of the lid part (22) and/or wherein the first wall section (30) of the bottom part (24) defines an entire bottom portion of the bottom part (24), and wherein the pharmaceutical carrier (20) is designed such that a ratio of a lateral extension of the lid and bottom part (22, 24) to a height of the assembled lid and bottom parts (22, 24) is >1, preferably ≥1.4, more preferably ≥1.5, even more preferably ≥2, most preferably ≥2.4 and in particular ≥2.5.

In the following, the present invention as defined in the embodiments is further illustrated by the following examples, which are not intended to limit the scope of the present invention. All references cited herein are explicitly incorporated by reference.

### EXAMPLES

### Example 1

In a first screening round, different primary matrix formers were investigated. In order to be suitable for use as a primary matrix former in a formulation for injection moulding of a pharmaceutical carrier, the primary matrix former in itself or in combination with other carrier shell components must be processable via hot melt extrusion and injection moulding, provide adequate mechanical strength to the capsule shell and afford appropriate drug release.

For example, Eudragit E was tested at different temperatures of the bulk mass and of the mould. It was observed that Eudragit E displayed sticky behaviour, resulting in blocking of the mould even when formulated with plasticizers and other secondary matrix formers. Maltodextrin caramelized, and hydroxy propyl cellulose (HPC) resulted in foaming and blocking during injection moulding. Also Povacoat could not be suitably applied in the injection moulding process. Starches alone, e.g. pea starch, hydroxy propyl pea starch, potato starch, maize or wheat starch yielded a poor strand quality after hot melt extrusion. In case of potato starch, the mass was gum-like, and for the other starches retrogradation lead to brittleness.

Surprisingly, polyvinyl alcohol was found to be workable, in particular in a mixture with stearic acid and propan-2-glycol (propylene glycol). Early formulations comprised 61.4% (w/w) polyvinyl alcohol PVOH (4-88), 21.9% (w/w) talc, 2% (w/w) stearic acid and 15% (w/w) propan-2-glycol. We also tested this early formulation without talc and found it to work well. However, dissolution data gave a lag time of up to 10 minutes.

In order to render the PVOH formulation not only suitable for injection moulding, but in particular for injection moulding of a pharmaceutical carrier, the inventors tested the PVOH formulation in combination with different pore formers/disintegration aids. The test formulations were evaluated for processability in injection moulding, stability, mechanical properties, and dissolution rate.

Stability was tested by storing pharmaceutical carriers prepares by injection moulding from the test formulation at 25° C. and 60% room humidity, 30° C. and 75% room humidity, or 40° C. and 75% room humidity. The carriers were examined by optical inspection after 3 and 6 months for deformations, phase separations, and the like.

The dissolution rate of a capsule can be determined using the ‘assay for immediate release’ as described in the US Pharmacopeia, section <711> from 2011. The assay uses the USP apparatus II (paddle) and Fasted state simulating gastric fluid (FasSGF; commercially available) at 37° C. and 50 rpm with n=3 capsules. In one embodiment, the pharmaceutical carrier exhibits a dissolution rate of at least 30%, preferably at least 40%, more preferably at least 50%, more preferably at least 60%, more preferably at least 65%, and most preferably at least 70% drug substance within 5 minutes; e.g. using propanolol.HCl as the test substance.

Mechanical strength determined via puncture force and snap open measurements can be tested for using standardized assays generally known in the art. Puncture force was measured by a texture analyzer equipped with a flat faced pin of defined surface area, which exerts pressure on the carrier part until the material fails and is punctured. From the pressure and the pin surface area the force is calculated. Mechanically strong formulations could also be measured for ‘snap-open’ force. In these measurements empty closed carriers were placed in a standard tablet crusher, and the force measured at which the carriers snap open. The results are shown in the following table.

| | Processability | | | |
|---|---|---|---|---|
| | in injection | | Dissolution | Puncture |
| Excipient | moulding | Stability | rate | force |
| Isomalt | + | − | o | o |
| Maltodextrin | −− | not | not | not |
| | | determined | determined | determined |
| NaCl | o | o | − | − |
| KCl | o | o | − | + |
| Wheat starch | + | + | + | + |
| Maize starch | ++ | + | + | + |
| Sorbitol | o | − | o | o |
| CaCO₃ | − | not | not | not |
| | | determined | determined | determined |
| Citric acid | −−− | not | not | not |
| | | determined | determined | determined |
| DCP | − | not | not | not |
| | | determined | determined | determined |
| NaHCO₃ | − | not | not | not |
| | | determined | determined | determined |
| Xylitol | o/− | o | − | o |
| Mannitol | o | o | − | o |

These foregoing screening studies showed that PVOH (4-88) in combination with either maize starch or wheat starch had the most suitable properties for injection moulding of a pharmaceutical carrier. Starting from this initial data, several formulations were tested, and found to be suitable for preparing a pharmaceutical carrier by injection moulding. Formulations (A) to (O) are examples of such formulations, which were found to be suitable.

| | Formulation | | | | | | | | |
|---|---|---|---|---|---|---|---|---|---|
| | A | B | C | D | E | F | G | H | I |
| Component | % (w/w) | | | | | | | | |
| PVOH (4-88) | 48.6 | 46.9 | 56.3 | 36.6 | 27.1 | 47.8 | 48.8 | 37.8 | 38.9 |
| Maize Starch | 40.1 | 40 | 30.5 | 50.3 | 59.9 | 40.2 | 40.2 | 50.2 | 50.1 |
| Stearic acid | 0.3 | 2 | 2 | 2 | 2 | 2 | 2 | 2 | 2 |
| Propan-2-glycol | 10 | 10 | 10 | 10 | 10 | 9 | 8 | 9 | 8 |
| Excipients | 1 | 1.1 | 1.1 | 1 | 1 | 1 | 1 | 1 | 1 |

| | Formulation | | | | | |
|---|---|---|---|---|---|---|
| | J | K | L | M | N | O |
| Component | % (w/w) | | | | | |
| PVOH (4-88) | 41.9 | 57.7 | 58.8 | 59.9 | 61 | 61.9 |
| Maize Starch | 50 | 30.2 | 30.2 | 30 | 30 | 30 |
| Stearic acid | 2 | 2 | 2 | 2 | 2 | 2 |
| Propan-2-glycol | 5 | 9 | 8 | 7 | 6 | 5 |
| Excipients | 1 | 1 | 1 | 1 | 1 | 1 |

Further testing revealed a particular balanced Formulation (1), which is also used in the following experiments.

Formulation (1)

| Component | % (w/w) |
|---|---|
| PVOH (4-88) | 62 |
| Maize Starch | 30 |
| Stearic acid | 2 |
| Propan-2-glycol | 5 |
| Excipients | 1 |

Formulation (1) can suitably applied in producing pharmaceutical carriers as exemplified in FIGS. 1-3.

### Example 2

12 mm standard Prescido™ carriers as shown in FIG. 1 were prepared by injection moulding using above Formulation (1).

For comparison, 12 mm standard Prescido™ carriers as shown in FIG. 1 were prepared by injection moulding using a formulation comprising polyethylene oxide (PEO) as the matrix former instead of PVOH. The PEO formulations comprised 73.5% (w/w) PolyOx N10, 20% (w/w) PolyOx N80, 5% (w/w) talc, and 1.5% (w/w) excipients.

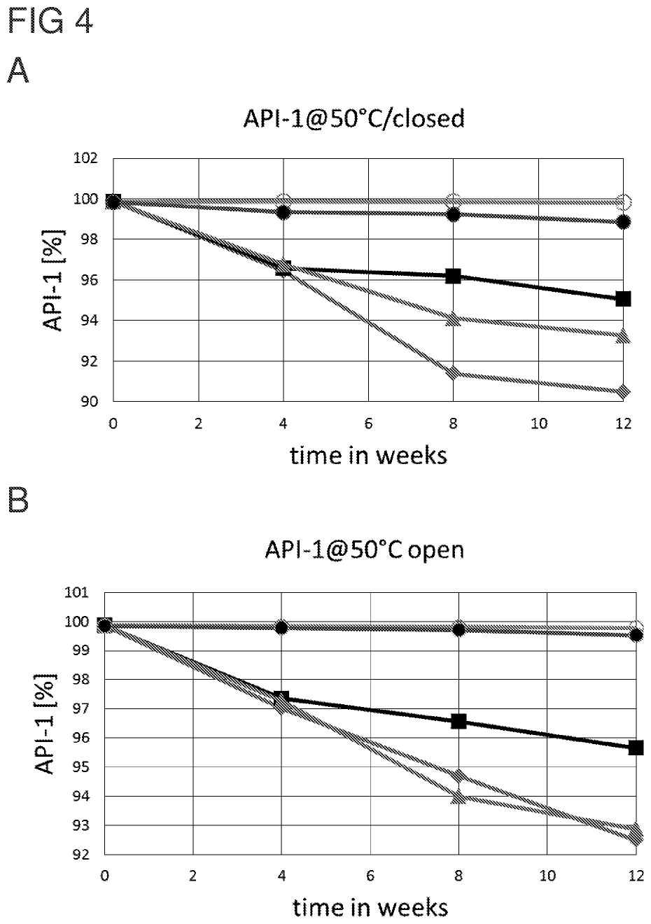

The carriers were then each filled with identical amounts of model neat API (API-1), and stored under stressed conditions at 50° C./75% RH for twelve weeks in closed and in open state. Samples are taken after 4, 8 and twelve weeks and tested for degradation of API-1, expressed as % API-1 as compared to API-1 alone. The results are shown in FIG. 4.

FIG. 4A shows the stability study for the carriers in the closed state. The control (API-1 alone) is the uppermost graph with open circles. The PVOH carriers (filled circles) maintained the stability of API-1 over the twelve weeks of storage under stressed conditions. At the end of the study, the carriers still contained well more than 98% API-1, even though the PVOH formulation did not contain any antioxidants. In contrast, the PEO carriers (1) (filled diamonds), (2) (filled squares), or (3) (filled triangles) showed considerably higher degradation of API-1 (less than 97% after 4 weeks; less than 96% after 12 weeks).

FIG. 4B shows the stability study for the carriers in the open state. The control (API-1 alone) is the uppermost graph with open circles. The PVOH carriers (filled circles) maintained the stability of API-1 over the twelve weeks of storage under stressed conditions. At the end of the study, the carriers still contained well more than 99% API-1. In contrast, the PEO carriers (1) (filled diamonds), (2) (filled squares), or (3) (filled triangles) showed considerably higher degradation of API-1 (less than 98% after 4 weeks; less than 97% after 8 weeks; less than 96% after 12 weeks).

Surprisingly, it was found that pharmaceutical carriers prepared from the PVOH formulation for injection moulding of the present disclosure maintain the stability of API-1 better than comparable carriers prepared from PEO formulations for injection moulding.

### Example 3

12 mm standard Prescido™ carriers as shown in FIG. 1 were prepared by injection moulding using above Formulation (1).

For comparison, 12 mm standard Prescido™ carriers as shown in FIG. 1 were prepared by injection moulding using the historic PVOH formulation comprising 61.4% (w/w) polyvinyl alcohol PVOH (4-88), 21.9% (w/w) talc, 2% (w/w) stearic acid, and 15% (w/w) propan-2-glycol.

In addition, 12 mm standard Prescido™ carriers as shown in FIG. 1 were prepared by injection moulding using a PEO formulation comprising 73.5% (w/w) PolyOx N10, 20% (w/w) PolyOx N80, 5% (w/w) talc, and 1.5% (w/w) excipients including antioxidants.

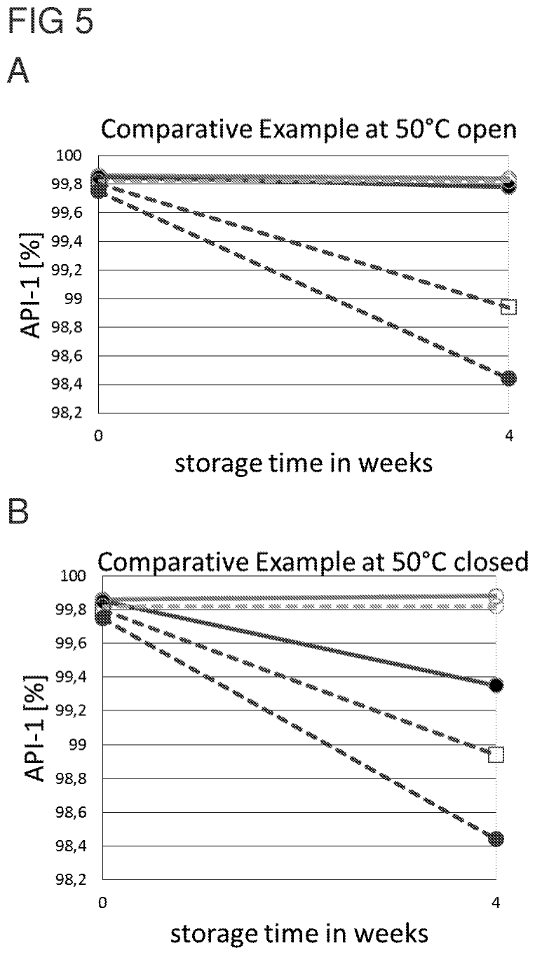

The carriers were then each filled with identical amounts of model neat API (API-1), and stored under stressed conditions at 50° C./75% RH for four weeks in closed and in open state. Samples are taken after 4 weeks and tested for degradation of API-1, expressed as % API-1 as compared to API-1 alone. The results are shown in FIG. 5.

FIG. 5A shows the stability study for the carriers in the open state. The controls (API-1 alone) are shown as the uppermost dashed and solid lines the with each open circles. The PVOH carriers prepared from formulation (1) (solid line, filled circles) maintained the stability of API-1 over the four weeks of storage under stressed conditions. At the end of the study, the carriers still contained well more than 99.7% API-1. In contrast, the PEO carriers (dashed line, open squares) and the PVOH carriers prepared from the historic formulation (dashed line, filled circles) showed higher degradation of API-1 (less than 99% after 4 weeks).

FIG. 5B shows the stability study for the carriers in the closed state. The controls (API-1 alone) are shown as the uppermost dashed and solid lines the with each open circles. The PVOH carriers prepared from formulation (1) (solid line, filled circles) maintained the stability of API-1 over the four weeks of storage under stressed conditions. At the end of the study, the carriers still contained well more than 99.3% API-1. In contrast, the PEO carriers (dashed line, open squares) and the PVOH carriers prepared from the historic formulation (dashed line, filled circles) showed higher degradation of API-1 (less than 99% after 4 weeks).

This comparative examples shows that the advantageous effects are due to the combination of PVOH and maize starch, and not due to the selection of PVOH as the primary matrix former as such.

### Example 4

12 mm standard Prescido™ carriers as shown in FIG. 1 were prepared by injection moulding using above Formulation (1).

For comparison, 12 mm standard Prescido™ carriers as shown in FIG. 1 were prepared by injection moulding using a formulation comprising polyethylene oxide (PEO) as the matrix former instead of PVOH. The PEO formulations comprised 73.5% (w/w) PolyOx N10, 20% (w/w) PolyOx N80, 5% (w/w) talc, and 1.5% (w/w) excipients.

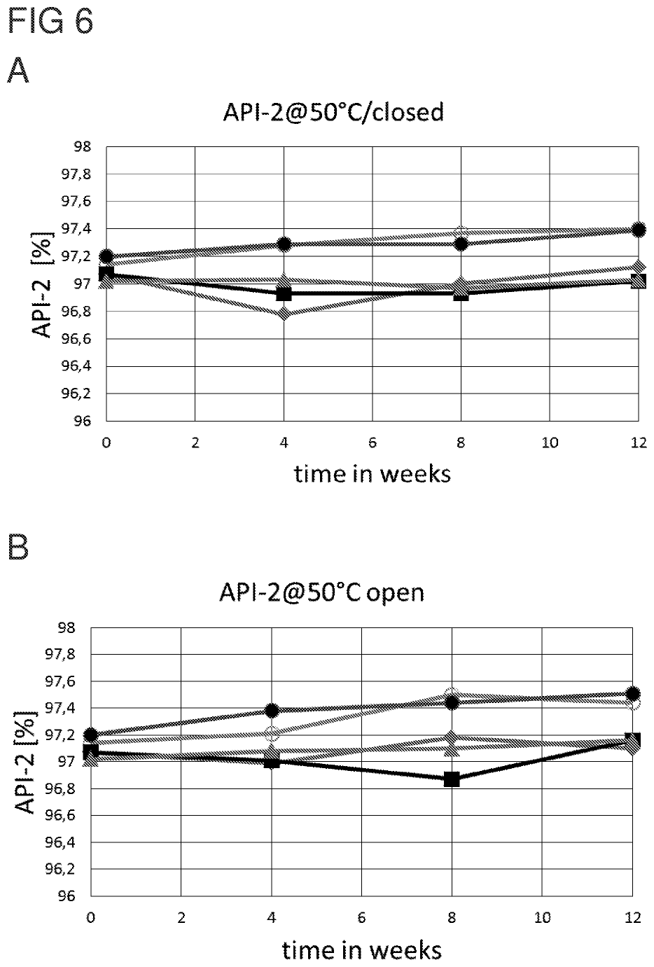

The carriers were then each filled with identical amounts of a different model neat API (API-2), and stored under stressed conditions at 50° C./75% RH for twelve weeks in closed and in open state. Samples are taken after 4, 8 and twelve weeks and tested for degradation of API-2, expressed as % API-2 as compared to API-2 alone. The results are shown in FIG. 6.

FIG. 6A shows the stability study for the carriers in the closed state. The control (API-2 alone) is the graph with open circles. The PVOH carriers (filled circles) maintained the stability of API-2 over the twelve weeks of storage under stressed conditions. At the end of the study, the carriers contained amounts of API-2 comparable to the control. In contrast, the PEO carriers (1) (filled diamonds), (2) (filled squares), or (3) (filled triangles) showed slight degradation of API-2.

FIG. 6B shows the stability study for the carriers in the open state. The control (API-2 alone) is the graph with open circles. The PVOH carriers (filled circles) maintained the stability of API-2 over the twelve weeks of storage under stressed conditions. At the end of the study, the carriers contained amounts of API-2 comparable to the control. In contrast, the PEO carriers (1) (filled diamonds), (2) (filled squares), or (3) (filled triangles) showed slight degradation of API-2.

Surprisingly, it was found that pharmaceutical carriers prepared from the PVOH formulation for injection moulding of the present disclosure maintain the stability of API-2 better than comparable carriers prepared from PEO formulations for injection moulding.

### Example 5

Pharmaceutical carriers were prepared by injection moulding using Formulation (1), comprising 62% (w/w) polyvinyl alcohol PVOH (4-88), 30% (w/w) maize starch, 2% (w/w) stearic acid, 5% (w/w) propan-2-glycol, and 1% excipients.

As a comparative example, pharmaceutical carriers were prepared by injection moulding using a PVOH formulation comprising 65% (w/w) polyvinyl alcohol PVOH (4-88), 12% (w/w) propan-2-glycol, 15% (w/w) CaCO₃, 5% (w/w) talc, 2% (w/w) stearic acid, and 1% (w/w) excipients.

As still another comparative example, hard gelatine capsules were used. The capsules were each directly filled with propranolol.HCl, without addition of further excipients. The dissolution rate of the capsules was determined using the ‘assay for immediate release’ as described in the US Pharmacopeia, section <711> from 2011. The assay uses the USP apparatus I (basket) and Fasted state simulating gastric fluid (FasSGF; commercially available) at 37° C. and 100 rpm in a volume of 900 ml. The following data was acquired:

| | | PVOH comparative | Hard gelatine |
|---|---|---|---|
| TP | Formulation (1) | example | capsules |
| (Min) | Release (%) | Release (%) | Release (%) |
| 0 | 0 | 0 | 0 |
| 5 | 7.97 | −0.30 | 60.5 |
| 10 | 91.45 | 23.04 | 90.4 |
| 15 | 96.96 | 47.54 | 94.7 |
| 30 | 100.66 | 97.70 | 98.4 |
| 45 | 101.20 | 102.06 | 98.6 |
| 60 | 101.28 | 102.81 | |

Results show that the dissolution profile of the injection moulded carrier prepared from the formulation of the present disclosure is similar to industry standard hard gelatin capsules (HGC), giving rapid and total release of API. In contrast, capsules prepared from PVOH, and in this particular example formulated with a hygroscopic salt (calcium carbonate), display a significant lag time. After 15 minutes, less than 50% of API is released.

1. A formulation for injection moulding of a pharmaceutical carrier, wherein the formulation comprises 27-85% (w/w) of polyvinyl alcohol; and 10-60% (w/w) of a disintegration aid selected from maize starch, wheat starch, and combinations thereof; and optionally one or more excipients.

2. The formulation of claim 1, wherein said polyvinyl alcohol is polyvinyl alcohol (4-88).

3. The formulation of claim 1 or 2, wherein the formulation comprises 35-82% (w/w) polyvinyl alcohol, preferably 40-80% (w/w), more preferably 45-75% (w/w), more preferably 50-70% (w/w), more preferably 55-68% (w/w), more preferably 60-65% (w/w), and most preferably about 62% (w/w) of said polyvinyl alcohol.

4. The formulation of any one of claims 1 to 3, wherein the formulation comprises 15-55% (w/w), preferably 17.5-50% (w/w), preferably 20-45% (w/w), preferably 22.5-40% (w/w), preferably 25-37.5% (w/w), preferably 27.5-35% (w/w), even more preferably 28-32% (w/w), and most preferably about 30% of said disintegration aid; in particular wherein the disintegration aid is maize starch.

5. The formulation of any one of claims 1 to 4, wherein the excipient is at least one selected from the list consisting of lubricant, process aid, colorant, opacifier, filler, and glidant.

6. The formulation of claim 5, wherein the formulation comprises 0.3-3.0% (w/w) of a lubricant, preferably 0.5-2.8% (w/w), more preferably 1.0-2.6% (w/w), more preferably 1.2-2.6% (w/w), more preferably 1.4-2.4% (w/w), more preferably 1.6-2.2% (w/w), more preferably 1.8-2.1% (w/w), and most preferably about 2% (w/w) of a lubricant; in particular wherein the lubricant is stearic acid or one of its salts.

7. The formulation of any one of claims 5-6, wherein the formulation comprises 5-14% (w/w) of a process aid, preferably 5-12% (w/w), more preferably 5-10% (w/w), more preferably 5-8% (w/w), even more preferably 5-6% (w/w), and most preferably about 5% (w/w) of a process aid; in particular wherein the process aid is propan-2-glycol.

8. The formulation of any one of claims 1 to 7, comprising 60-65% (w/w) polyvinyl alcohol (4-88), 28-32% (w/w) of maize starch, 1.8-2.1% (w/w) of stearic acid, and 5-6% (w/w) of propan-2-glycol.

9. A method of producing a pharmaceutical carrier, comprising the steps of
(a) melting a formulation according to any one of claims 1 to 8, and
(b) injecting the melt into a mould, and
(c) optionally cooling the injected melt and optionally ejecting the moulded material, and
(d) optionally sorting the carrier parts by mould cavity.

10. The method of claim 9, wherein the pharmaceutical carrier (20) is a capsule, and at least one lid part (22) and at least one bottom part (24) is formed.

11. The method of claim 10, wherein at least one of the lid part (22) and the bottom part (24) has a first wall section (26, 30) with a thickness of 180-250 μm, and a second wall section (28, 32) with a thickness of 350-450 μm, and wherein the first wall section (26) of the lid part (22) defines an entire top portion of the lid part (22) and/or wherein the first wall section (30) of the bottom part (24) defines an entire bottom portion of the bottom part (24).

12. The method of claim 10 or claim 11, wherein the lid part (22) and the bottom part (24) are connected to each other by a complementary closing mechanism (34);
wherein the closing mechanism (34) comprises a first snap part (36) which projects from the second wall section (32) of the bottom part (24) so as to face and to interact with a second snap part (38) which projects from the second wall section (28) of the lid part (22);
wherein the first snap part (36) comprises a projection (37) adapted to engage with a corresponding projection (39) provided on the second snap part (38) so as to counteract separation of the first snap part (36) and the second snap part (38) and thus separation of the lid part (22) and the bottom part (24);
wherein the projection (37) provided on the first snap part (36) tapers in a direction of a free end of the first snap part (36) so as to form a first inclined engagement surface (45) adapted to engage with a second inclined engagement surface (47) formed on the projection (39) provided on the second snap part (38) which tapers in a direction of a free end of the second snap part (36).

13. The method of any one of claims 9 to 12, wherein the pharmaceutical carrier (20) is designed such that a ratio of a lateral extension of the lid and bottom part (22, 24) to a height of the assembled lid and bottom parts (22, 24) is >1, preferably ≥1.4, more preferably ≥1.5, even more preferably ≥2, most preferably ≥2.4 and in particular ≥2.5.

14. A pharmaceutical carrier produced by the method of any one of claims 9 to 13 using the formulation of any one of claims 1 to 8, comprising
a lid part (22) and
a bottom part (24),
wherein each of the lid part (22) and the bottom part (24) has a first wall section (26, 30) with a thickness of 180-250 μm and a second wall section (28, 32) with a thickness of 350-450 μm, and wherein the first wall section (26) of the lid part (22) defines an entire top portion of the lid part (22) and/or wherein the first wall section (30) of the bottom part (24) defines an entire bottom portion of the bottom part (24).
in particular wherein the pharmaceutical carrier allows for a dissolution rate of at least 80%, more preferably at least 85%, more preferably at least 90%, and most preferably at least 95% drug substance within 15 minutes; for fast dissolving compounds when tested using the ‘assay for immediate release’ described in the US Pharmacopeia 2011, section <711>

15. The pharmaceutical carrier of claim 14, wherein the pharmaceutical carrier is filled with an active pharmaceutical ingredient (API) and optionally comprising at most 5% (w/w) of an additive.
